(19) United States
(12) Patent Application Publication
(10) Pub. No.: US 2014/0079815 A1
(43) Pub. Date: Mar. 20, 2014

(54) COMPOSITIONS FOR STIMULATING HAIR GROWTH AND PREVENTING HAIR LOSS

(71) Applicant: Edward Amos KENNEDY, Marietta, PA (US)

(72) Inventor: Edward Amos KENNEDY, Marietta, PA (US)

(21) Appl. No.: 13/622,406

(22) Filed: Sep. 19, 2012

(51) Int. Cl. A61K 36/28; A61P 17/14

(52) U.S. Cl. 424/721

(57) ABSTRACT

A composition for promoting hair growth, slowing hair loss, and for preventing or minimizing hair loss that do not suffer from the deficiencies of the prior art. One exemplary embodiment is directed to a topical formulation for hair, the formulation combining arnica and aqua ammonia to promote hair growth and hair loss diminution.

### FIELD OF THE INVENTION

The present invention relates to compositions, methods and products for promoting hair growth, slowing hair loss, preventing hair loss, or minimizing hair loss. In particular, the present invention relates to compositions and products administered topically containing arnica for promoting hair growth, slowing hair loss, preventing hair loss, or minimizing hair loss.

### BACKGROUND OF THE INVENTION

Hair loss or alopecia may be caused by a variety of factors including heredity, hormonal deficiencies or imbalances, diet, stress, chemotherapy or aging. The desire to maintain or regain head hair has led to continuing efforts throughout history to discover compositions and methods for stimulating hair growth and for preventing or minimizing hair loss.

For many years, the pharmaceutical industry, the nutraceutical industry, and the cosmetic industry have been researching and developing compositions that will cure or prevent hair loss or promote hair growth.

There are surgical and non-surgical solutions to address the problem of hair loss. Non-surgical options for treating hair loss include a variety of pharmaceutical and nutraceutical topical and/or oral treatments that promote hair re-growth and/or prevent further hair loss. For example, topical minoxidil, commonly known as ROGAINE, causes hair growth when applied to the scalp and slows the rate of hair loss in some individuals by stimulating hair follicles. Finasteride, commonly known as PROPECIA is a drug that is taken orally to treat androgenic alopecia by blocking the formation of DHT. The problem with treating hair loss with pharmaceutical drugs is the potential side effects of such drugs. Minoxidil may cause low blood pressure, increase in heart rate, weight gain due to water retention, and the scalp may become inflamed. Finasteride may cause genital deformities in male infants, impotence, decreased libido, hives or rash, and swelling.

U.S. Pat. No. 5,422,100 issued Jun. 6, 1995, to Eliaz et al. discloses methods and products for promoting hair growth, preventing or minimizing hair loss, enhancing or restoring hair color and treating other hair and skin afflictions. The methods of the '100 patent include topical applications of the products to the skin or hair follicles being treated. The product of the '100 patent includes a treating agent selected form the class of chemicals consisting of anol, anethole, analogs of the above, polymers of the above, and mixtures of the above with various mixture of these chemicals being found in herb families including umbelliferae, magnoliaceae, labiatae and rutaceae. The '100 invention preferably selects the treatment agent from the class of herbs consisting of Foeniculum vulgares (fennel seed), Pimpinella anisum (anise), Carum carvi (caraway seeds) and mixtures thereof with each other and/or other herbs.

U.S. Pat. No. 5,514,672 issued May 7, 1996, to Bazzano discloses an oral administration or topical application to the skin, hair, and/or hair follicle of effective amounts of retinoid, particularly retinoic acid to increase rate of hair growth, stimulation of hair follicles to produce to produce new hair growth, prolongation of the anagen phase of the hair cycle, and conversion of vellus hair to growth as terminal hair and treatment of alopecia due to organic dysfunction of the hair follicle. The retinoid of the '627 patent may be administered or applied alone or with other adjuctive compounds including vitamins, such as Vitamin D.sub.3, hormones, antiandrogens and/or vasodiliators.

U.S. Pat. No. 5,629,002 issued May 13, 1997, to Weuffen et al. discloses cosmetic or pharmaceutical preparations for improving the quality and stimulating the growth of hair. The preparations of the '002 patent are based on A) alkali metal, alkaline earth metal and/or ammonium salts of thiocyanic acid in combination with B) at least one component selected from estrogens, sulfur, sulfide ions, vasodilators, skin-active vitamins, inorganic selenium compounds, amino acids, protein hydrolyzates and carboxylic acids physiologically occurring in the skin or mixtures thereof. The preparations may optionally be in admixture with per se known auxiliary and carrier materials for hair cleaning and hair care agents. A synergistic improvement in the quality of hair and stimulation of hair growth is observed by combining the alkali metal, alkaline earth metal and/or ammonium slats of thiocyanic acid with at least one of the component B materials.

U.S. Pat. No. 5,674,510 issued Oct. 7, 1997, to DiTucci discloses a hair treatment solution capable of acting as a cosmetic, reducing alopecia, eliminating alopecia, increasing hair growth or any combination thereof. The solution of the '510 patent comprises garlic powder, brewer's yeast, grapefruit juice, acetic acid and kelp.

U.S. Pat. No. 6,103,272 issued Aug. 15, 2000, to Keeney discloses a topical solution, and method of making the same, for promoting hair growth which includes a colloidal silver solution. An aloe vera gel containing allantoin, arnica flowers, comfrey leaves, horsetail herbal extract, jojoba, collogen, elastin, saponins, chamomile flowers, elkweed, jaborandi leaves, napca and rosemary leaves, may be added to enhance the effectiveness of the topical applications of colloidal metal of the present invention.

U.S. Pat. No. 7,205,009 issued Apr. 17, 2007, to Stass discloses an herbal composition for the re-growth of human hair includes the combination of hypercium perforatum, veronica beccabunga, veronica officinalis, and equisetum arvense wherein the combination is infused in water and adapted to be applied to the scalp of a user.

Thus, in spite of the advancements in the prior art, there is a need in the art for compositions for promoting hair growth, slowing hair loss, and for preventing or minimizing hair loss that do not suffer from the deficiencies of the prior art, and for methods of making and using such compositions.

### SUMMARY OF THE INVENTION

It is an object of the present invention to provide for compositions for promoting hair growth, slowing hair loss, and for preventing or minimizing hair loss that do not suffer from the deficiencies of the prior art.

One exemplary embodiment is directed to a topical formulation for hair, the formulation combining arnica and aqua ammonia to promote hair growth and hair loss diminution.

One exemplary embodiment is directed to a topical formulation for hair, the formulation combining arnica, aqua ammonia, alcohol and water to promote hair growth and hair loss diminution.

One exemplary embodiment is directed to a topical formulation for hair, the formulation combining arnica, aqua ammonia and alcohol to promote hair growth and hair loss diminution.

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

FIG. 1 is a photograph taken prior to treatment illustrating the scalp prior to hair growth.

FIG. 2 is a photograph taken at about the end of the first week of treatment illustrating hair growth.

FIG. 3 is a photograph taken at about the end of the second week of treatment illustrating hair growth.

FIG. 4 is a photograph taken at about the end of the third week of treatment illustrating hair growth.

FIG. 5 is a photograph taken at about the end of the second month of treatment illustrating hair growth.

FIG. 6 is a photograph taken at about the end of the third month of treatment illustrating hair growth.

FIG. 7 is a photograph taken at about the end of the fourth month of treatment illustrating hair growth.

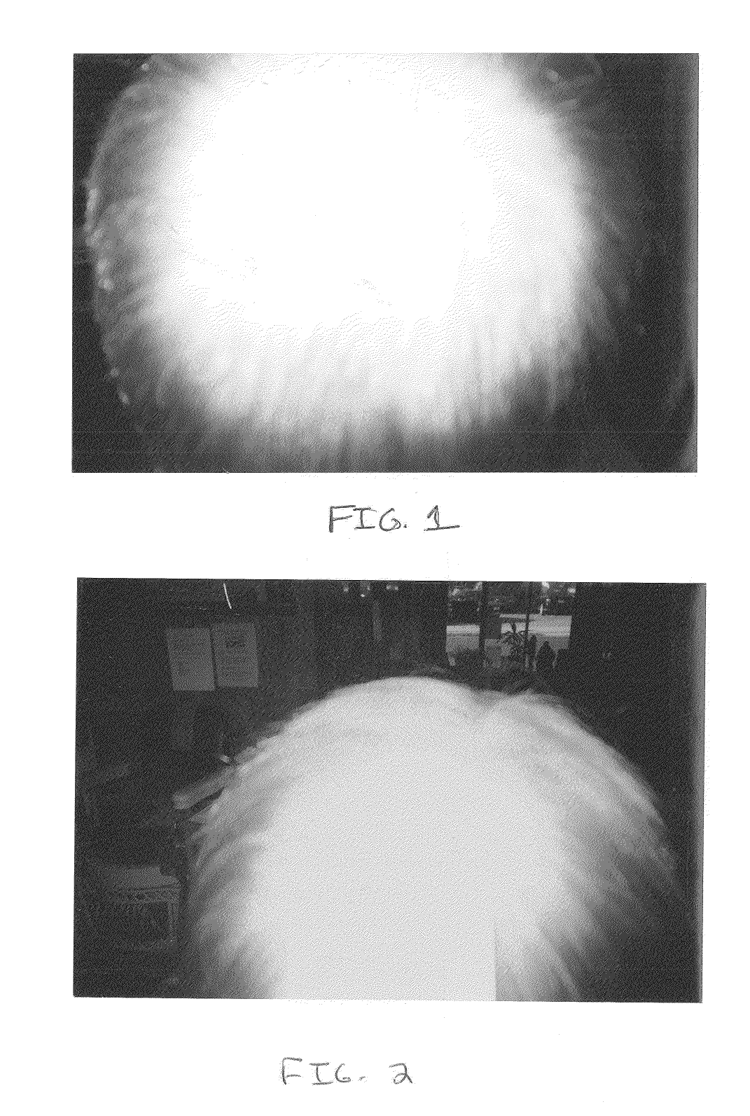

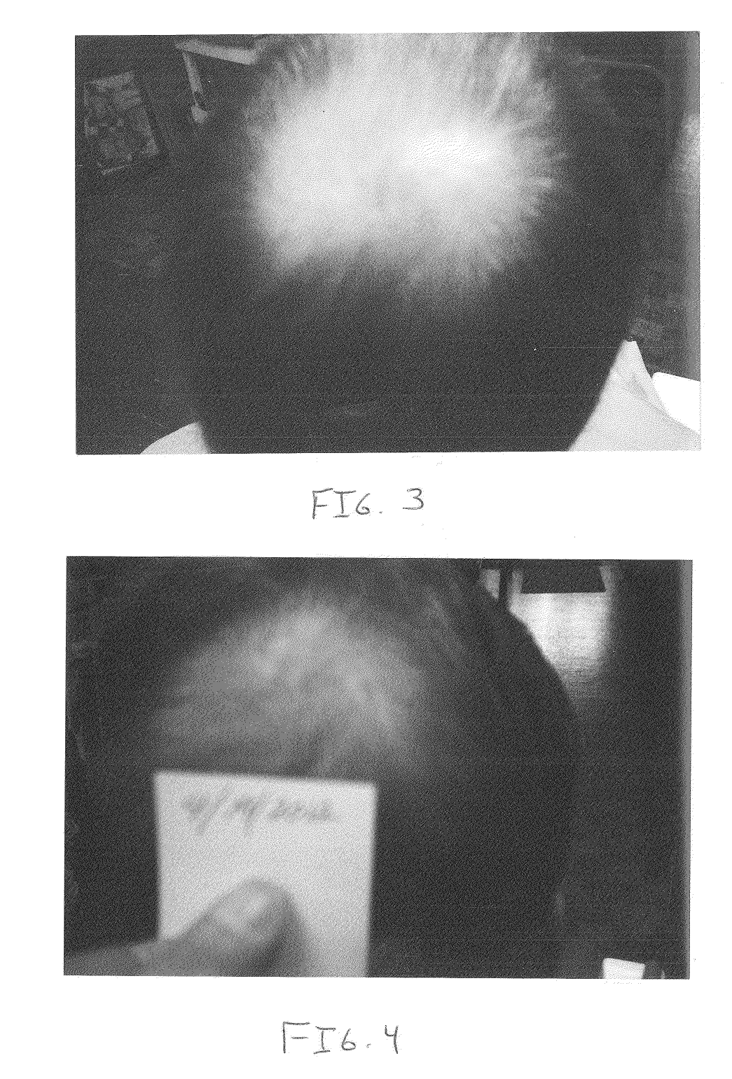

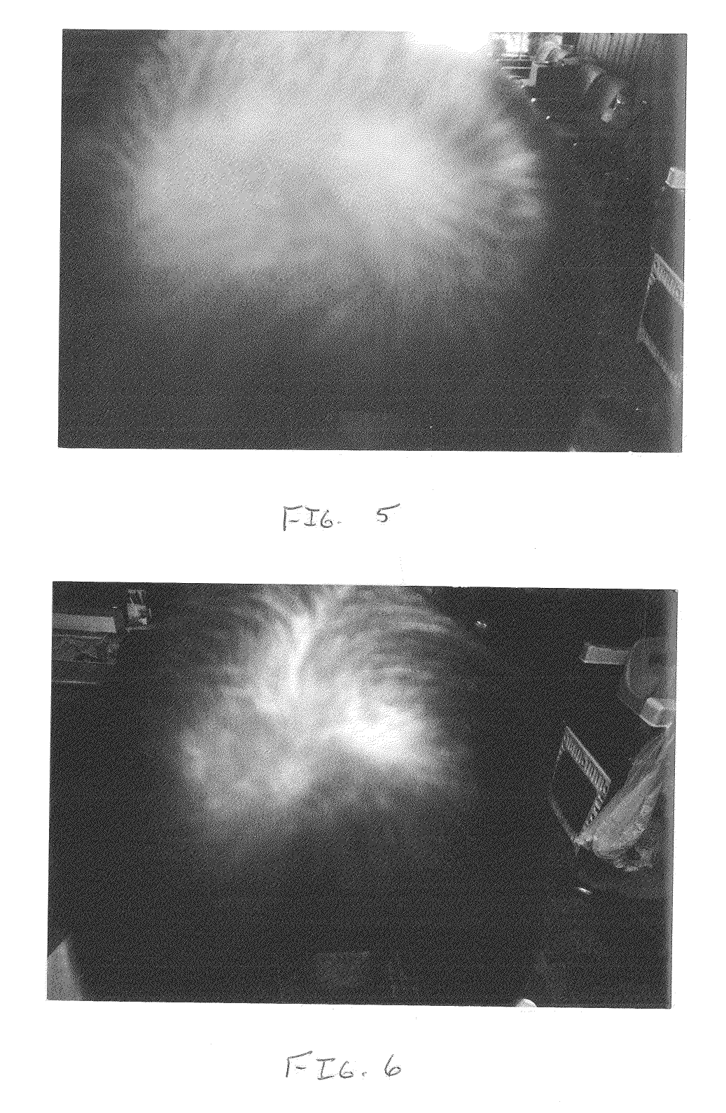

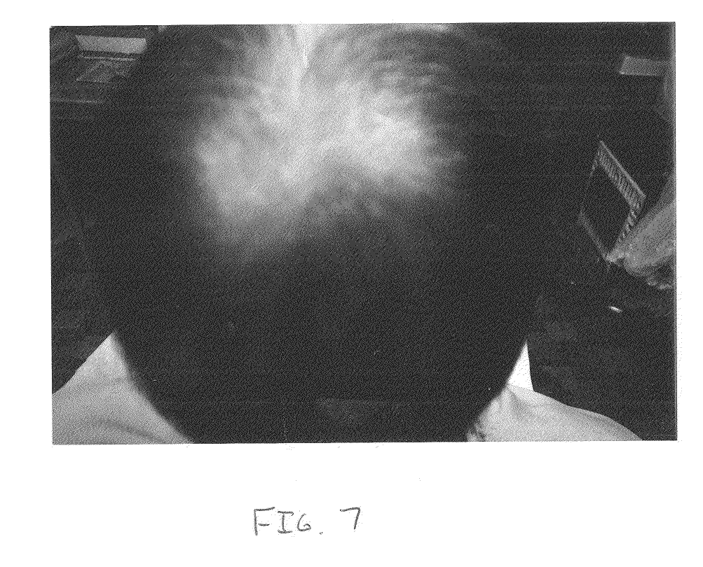

### DETAILED DESCRIPTION OF THE INVENTION

In accordance with the present invention, it has been found that topical administration of arnica in combination with aqua ammonia can be used to increase hair growth in patients suffering from hair loss, to slow hair loss, or to prevent or minimize hair loss. The use of the combination has also been found to prevent or eliminate grey hair.

Arnica is a genus with approximately 30 perennial, herbaceous species, belonging to the Asteraceae family. Arnica (Arnica montana) has been used for medicinal purposes since the 1500s and is still popular today. Applied to the skin as a cream, ointment, liniment, salve, or tincture, arnica has been used topically for a wide range of conditions, including bruises, sprains, muscle aches, wound healing, superficial phlebitis, joint pain, inflammation from insect bites, and swelling from broken bones. It is commonly used for injuries such as sprains and bruises. However, the benefits of a composition using Arnica for hair growth purposes have not been addressed in the past. The present invention specifically aims at the demonstration of the important hair growth stimulation and hair loss reduction properties of a topical application of arnica in combination with aqua ammonia.

Aqua ammonia, also known as ammonium hydroxide, ammonia water, ammonical liquor, ammonia liquor, ammonia solution, aqueous ammonia, or simply ammonia, is a solution of ammonia in water. It can be denoted by the symbols NH3(aq). As a food additive, aqua ammonia is used as an antimicrobial. In the United States, aqua ammonia is classified by the Food and Drug Administration and is generally recognized as safe. Food treated with aqua ammonia can be found on grocery shelves. Aqua ammonia is also known to be used by aquarists for the purposes of fishless cycling. However, the benefits of a composition using aqua ammonia for hair growth purposes have not been addressed in the past. The present invention specifically aims at the demonstration of the important hair growth stimulation and hair loss reduction properties of a topical application of aqua ammonia in combination with arnica.

The two main components or active ingredients of the composition, arnica and aqua ammonia, operate concurrently with the synergetic ingredients in which they are incorporated to promote important hair growth and hair loss diminution. Synergetic ingredients used in the composition, include, but are not limited to, alcohol and distilled water. The distilled water and the alcohol provide a delivery system for the active ingredients as well as a dilution system. In addition, the use of alcohol, which evaporates relatively quickly, insures that the composition will only remain on the scalp of the user for a defined period of time. The rate of evaporation can be altered by adjusting the percentage of alcohol used in the composition.

The combination of these synergetic ingredients with the active ingredients of the arnica and aqua ammonia creates a synergetic effect on hair follicles that has been brought to light through this invention. Indeed, repeated topical applications of the composition in the effected region has been proven to be highly effective in increasing hair growth in individuals suffering from hair loss, or in preventing or delaying hair loss in individuals just beginning to lose their hair through.

An exemplary embodiment of the formulation of the composition resides in the combination of the arnica and the aqua ammonia with the alcohol and the distilled water. An exemplary method of treatment consists of one daily application of the composition over an extended period. Another exemplary method of treatment consists of one daily application of the composition for the first week, twice daily applications of the composition for the second week and one daily application of the composition for the third week and beyond, over an extended period. However, other application procedures may be used depending upon the desire to grow hair or to maintain current hair by preventing hair loss.

With proper application, the composition of the present invention has been shown to reinforce hair strength and general health, therefore decreasing hair loss, as well as inducing stimulation of hair follicles, resulting in subsequent hair growth, which may occur in as little as 2 weeks from the first treatment. Moreover, the composition has the quality of being highly natural and non-synthetic.

Table 1 provides the active and synergetic ingredients of one exemplary embodiment, and the proportions of the ingredients that comprise that exemplary composition.

TABLE 1

| | Allowable | Preferred |
|---|---|---|
| | Range | Range |
| Ingredient | (% by weight) | (% by weight) |
| Arnica | 1-3 | 1.5-2.8 |
| Aqua Ammonia | 2-15 | 3-11 |
| Alcohol | 40-50 | 42-48 |
| Distilled Water | 32-57 | 38.2-53.5 |

If an individual has sensitive skin or is undergoing treatments for cancer or the like, the percentage of the aqua ammonia can be reduced to prevent any potential damage to the scalp caused by the aqua ammonia. The reduction of the aqua ammonia may increase the time for the hair growth to occur, but hair growth will occur with the reduced aqua ammonia.

In alternate exemplary embodiments, other ingredients may be added to the composition. The ingredients may be added to change the fragrance, viscosity, color or other parameters of the composition. In addition, vitamins or other beneficial ingredients may be added without departing from the scope of the invention.

One representative formulation of an alternate exemplary embodiment is shown in Table 2. In this embodiment, elderflower water made from the Sambucus plant has been included in the formulation.

TABLE 2

| | Allowable | Preferred |
|---|---|---|
| | Range | Range |
| Ingredient | (% by weight) | (% by weight) |
| Arnica | 1-3 | 1.6-2.6 |
| Aqua Ammonia | 2-12 | 3-10 |
| Alcohol | 40-50 | 42-45 |
| Distilled Water | 29-53 | 30.1-48.1 |
| Elderflower Water | 4-6 | 4.3-5.3 |

The present invention will be more readily understood by referring to the following examples which are given to illustrate the invention rather than to limit its scope.

### EXAMPLE 1

Application: The composition described in Table 2 is applied 1 time a day on the region to treat in an amount sufficient to allow good penetration of the product in the hair roots and in the surrounding skin. The composition is massaged on the region to treat for at least 30 seconds. In some formulations, a lather may be evident when the formulation has been properly and thoroughly messaged into the region. It is indicated to avoid washing the treated region for the 3 to 4 days treatment period. If cleaning is necessary, organic shampoos or soaps should be used rather than chemical ones.

Results. The results for a representative subject are presented in Table 3. Similar results have been observed in other subjects as well. The positive response observed in the treated with the composition of the present invention constitutes direct evidence that the product is effective in increasing hair growth in patients suffering from hair loss and preventing or delaying hair loss in patients just beginning to lose their hair.

TABLE 3

| Time | Size of Bald Spot¹ |
|---|---|
| Prior to start of applications | 3¼ inches × 2¾ inches |
| 1 week after start of applications | 2¾ inches × 2¾ inches |
| 2 weeks after start of applications | 2⅝ inches × 2⅝ inches |
| 3 weeks after start of applications | 2½ inches × 2½ inches |
| 2 months after start of applications | 2½ inches × 2½ inches |
| 3 months after start of applications | 2½ inches × 2 inches |
| 4 months after start of applications | 2½ inches × 2 inches (thicker) |

¹The bald spot is measured in the X and Y directions and indicate the area in which not hair is present.

### EXAMPLE 2

Application: The composition described in Table 2 is applied 1 time a day on the region to treat in an amount sufficient to allow good penetration of the product in the hair roots and in the surrounding skin. The composition is massaged on the region to treat for at least 30 seconds. In some formulations, a lather may be evident when the formulation has been properly and thoroughly messaged into the region. It is indicated to avoid washing the treated region for the 3 to 4 days treatment period. If cleaning is necessary, organic shampoos or soaps should be used rather than chemical ones.

Results. The results are presented in Table 4. The positive response observed in the treated with the composition of the present invention constitutes direct evidence that the product is effective in increasing hair growth in patients suffering from hair loss and preventing or delaying hair loss in patients just beginning to lose their hair.

TABLE 4

| | | | Treatment | |
|---|---|---|---|---|
| Subject | Sex | Age | Term | Results |
| 1 | M | 42 | 2 months | Hair growth, discontinuance |
| | | | | of hair loss |
| 2 | M | 48 | 6 months | Hair growth, discontinuance |
| | | | | of hair loss |
| 3 | M | 45 | 6 months | Hair growth, discontinuance |
| | | | | of hair loss |
| 4 | M | 50 | 6 months | Hair growth, discontinuance |
| | | | | of hair loss |
| 5 | M | 42 | 4 months | Hair growth, discontinuance |
| | | | | of hair loss |
| 6 | M | 47 | 7 months | Hair growth, discontinuance |
| | | | | of hair loss |

In other exemplary embodiments, the formulation may be changed and various other ingredients may be added to change some of the characteristics of the formulation to create gels, mousses, shampoos, conditioners, rinses and the like. The application of these products may vary depending upon whether the user is trying to prevent the loss of hair or trying to grow hair.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A topical formulation for hair, the formulation comprising:
arnica and aqua ammonia combined to promote hair growth and hair loss diminution.

2. The formulation of claim 1, wherein the arnica comprises 1% to 3% by weight of the formulation.

3. The formulation of claim 2, wherein the arnica comprises 1.6% to 2.6% by weight of the formulation.

4. The formulation of claim 1, wherein the aqua ammonia comprises 2% to 12% by weight of the formulation.

5. The formulation of claim 4, wherein the arnica comprises 1% to 3% by weight of the formulation.

6. The formulation of claim 4, wherein the aqua ammonia comprises 3% to 10% by weight of the formulation.

7. The formulation of claim 6, wherein the arnica comprises 1.6% to 2.6% by weight of the formulation.

8. The formulation of claim 1, wherein the formulation further comprises alcohol, the alcohol comprises about 40% to 50% by weight of the formulation.

9. The formulation of claim 8, wherein the alcohol comprises 42% to 45% by weight of the formulation.

10. The formulation of claim 1, wherein the formulation further comprises distilled water, the distilled water comprises 29% to 53% by weight of the formulation.

11. The formulation of claim 10, wherein the distilled water comprises 30.1% to 48.1% by weight of the formulation.

12. The formulation of claim 1, wherein the formulation further comprises elderflower water, the elderflower water comprises 4% to 6% by weight of the formulation.

13. The formulation of claim 12, wherein the elderflower water comprises 4.3% to 5.3% by weight of the formulation.

14. A topical formulation for hair, the formulation comprising:
arnica, aqua ammonia, alcohol and water combined to promote hair growth and hair loss diminution.

15. The formulation of claim 14, wherein the arnica comprises 1.6% to 2.6% by weight of the formulation.

16. The formulation of claim 15, wherein the aqua ammonia comprises 3% to 10% by weight of the formulation.

17. The formulation of claim 16, wherein the alcohol comprises about 42% to 45% by weight of the formulation.

18. The formulation of claim 17, wherein the water is distilled water and comprises 30.1% to 48.1% by weight of the formulation.

19. The formulation of claim 18, wherein the formulation further comprises elderflower water, the elderflower water comprises 4.3% to 5.3% by weight of the formulation.

20. A topical formulation for hair, the formulation comprising:
arnica, aqua ammonia, and alcohol combined to promote hair growth and hair loss diminution.
